(19) United States
(12) Patent Application Publication
(10) Pub. No.: US 2014/0353189 A1
(43) Pub. Date: Dec. 4, 2014

(54) Disposable Holder for Special Medical Procedure Devices

(71) Applicant: Maria Lotosky-Compton, Webster, NY (US)

(72) Inventor: Maria Lotosky-Compton, Webster, NY (US)

(21) Appl. No.: 13/907,004

(22) Filed: May 31, 2013

(51) Int. Cl. A61B 19/02

(52) U.S. Cl. CPC A61B 19/0256; A61B 2019/0201
USPC 206/370; 206/363

(57) ABSTRACT

A disposable holder for wires, stents and other materials used in conjunction with specialized medical devices such as endoscopic, angiographic or angioplastic tools generally comprised of a panel having a plurality of pouches for storing and organizing the wires/stents/catheters associated with respective surgical tools. Preferably, the back side of the panel is equipped with a fastener for removably fastening the panel to a surgical cart or table. The surgical tools, when not in use, are wound and stored within the pouches thereby alleviating the possibility of entanglement of wires/stents/catheters or having the wires/stents/catheters touch or rest on the surgical room floor. In an alternative embodiment, each holder has a stand-alone pouch having a fastener.

### FIELD OF THE INVENTION

The present invention relates to a system and device for collecting and organizing surgical materials such as wires, cables or tubing, and more particularly, the present invention relates to a system and device for collecting and organizing surgical materials associated with various invasive medical tools, for instance those used during endoscopic. angiographic or angioplastic procedures such as endoscopic retrograde cholangio-pancreatography or vascular catheterization.

### BACKGROUND OF THE INVENTION

Medical technology continues to reduce the need for extensively invasive surgical procedures by developing more noninvasive procedures which require smaller incisions or by passing tools along the gastrointestinal (GI) tract. Surgical tools are passed into the body (through the incision or by oral administration into the GI tract) with direction and manipulation of those tools controlled externally by the doctor. This direction and manipulation typically uses a series of fine gauge guide wires. Operational tools required in the procedure may also be directed into the body and guided to the site of interest by a guide wire. As such, typical noninvasive medical procedures require a number of wires and other medical materials, with these wires and materials resident at least partial outside of the patient during a procedure. It is imperative that these wires and other materials remain organized so as to avoid creating a dangerous situation for the doctors and nurses within the operating room, as well as avoid injury to the patient by unintentional/erroneous manipulation of the device inside the body. Additionally, to reduce the risk of infection and to maintain a sterile field, these wires and materials should be organized and contained so as not to contact any non-sterile surface such as the operating room floor.

One example of a procedure particularly amenable to the device disclosed in the present invention is endoscopic retrograde cholangio-pancreatography (ERCP). ERCP is a diagnostic procedure for examining diseases of the liver, bile ducts and pancreas that uses a small scope to examine the stomach and intestines. ERCP combines the use of a flexible, lighted scope (endoscope) with X-ray pictures to examine the tubes that drain the liver, gallbladder, and pancreas. Indeed, ERCP can provide important information that cannot be obtained by other diagnostic examinations like an abdominal ultrasound, CT scan, or MRI, and also allows for therapeutic measures to be performed at the time of ERCP procedure, if indicated. The endoscope and is passed down from the mouth into and beyond the stomach to rest within the duodenum near the papilla of Vater, which is the opening of the small intestine leading to the pancreatic and bile ducts. A small plastic catheter (cannula) is passed through an open channel of the endoscope into the openings of the papilla, and into the bile ducts and/or the pancreatic duct. Contrast material (dye) is then injected and X-rays are taken of the bile ducts and the pancreatic duct. An open channel in the endoscope also allows other instruments to be passed through it in order to perform biopsies, to insert plastic or metal stents to relieve obstruction(s) of the bile ducts caused by cancer or scarring, and to perform incisions by using electrocautery.

Typically, ERCP is done in a hospital or surgical center with the patient staying overnight if the procedure further entails removal of gallstones or placement of a stent. The endoscope is administered orally and is directed to the examination site by a doctor. Movement of the distal tip of the endoscope within the duodenum, along with any medical devices fed within the body of the endoscope, is controlled externally at the scope's handle by the doctor.

Typically, at least one guide wire is inserted through the endoscope and is guided to the area of interest within the gut. The guide wire is used to direct endoscope tools to the proper location. The guide wire is thin gauge and flexible allowing it to traverse the ducts without overly irritating the internal surfaces of the organs. Alternatively, endoscope tools may come preloaded with guide wires where the tool is loaded into the endoscope with the guide wire advanced before the operative tool. Thus, in a test requiring various procedures and the use of more than one instrument within the endoscope, a large number of wires exist within the operating room.

Presently, there is no convenient way to collect and organize these wires. Additionally, the loose wires can easily become entangled leading to a dangerous work environment, potential error and/or prolonged examination times. Prolonged examinations require the use of additional anesthetics, increase the possibility for patient infection and unnecessarily tie up doctors thereby preventing them from treating other patients.

A second example of a specialized medical procedure which would benefit from the device of the present invention is that requiring vascular catheterization, such as angiography and angioplasty. Both angiography and angioplasty generally involve passing a catheter and associated guide wires into a blood vessel of the patient. A contrast agent or dye is directed into the blood vessel at the desire location of examination during angiography. An x-ray or other imaging modality then images the blood vessels containing the agent/dye. In angioplasty, a stent is generally passed through the vessel until reaching a blocked/narrowed site wherein the stent is placed to prop open the vessel thereby allowing more proper blood flow through the vessel. Again, in each of these techniques, the catheter (and stent in the case of angioplasty) is passed along the vessel through external control of a doctor using a system of guide wires. Thus, it is imperative that these wire remain organized and as sterile as possible during the procedure to minimize chances of infection as well as to avoid dangerous entanglements and prolonged operation times.

Thus, there is a need for a system which collects and organizes the many wires and other materials associated with medical examinations and procedures, such as those used with an endoscope and associated tools during ERCP, or those used with vascular techniques which require vascular catheterization or stenting, which overcomes the problems described above. The present invention addresses these and other needs.

### BRIEF SUMMARY OF THE INVENTION

In general, one embodiment of the present invention is directed to a disposable holder for wires, stents and other materials used in conjunction with specialized medical devices such as endoscopic, angiographic or angioplastic tools which overcomes the problems and deficiencies in the prior art. The disposable holder of one embodiment of the present invention is generally comprised of a panel having a plurality of pouches for storing and organizing the wires/stents/catheters/etc. associated with respective surgical tools. Preferably, the back side of the panel is equipped with a fastener for removably fastening the panel to a surgical cart or table. The surgical tools, when not in use, are wound and stored within the pouches thereby alleviating the possibility of entanglement of wires/stents/catheters/etc. or having the wires/stents/catheters/etc. touch or rest on the surgical room floor. In an alternative embodiment, each holder has a stand-alone pouch having a fastener.

Additional objects, advantages and novel features of the present invention will be set forth in part in the description which follows, and will in part become apparent to those in the practice of the invention, when considered with the attached figures.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a perspective view of an exemplary endoscope;

FIG. 2 is a schematic view of an endoscope tube performing an ERCP procedure;

FIG. 2A is an expanded view of the distal end of the endoscope tube showing a catheter;

FIG. 3 is a schematic view of an endoscope tube performing a biopsy of tissue during an ERCP procedure;

FIG. 4 is a schematic view of an endoscope tube place a stent during an ERCP procedure;

FIG. 5. is a schematic view of an endoscope tube performing a sphincterotomy during an ERCP procedure.

FIG. 6 is a perspective view of the disposable holder for endoscopic wires and stents of one embodiment of the present invention.

### DETAILED DESCRIPTION OF THE INVENTION

Preferred embodiments of a disposable holder for special medical procedure devices according to the present invention will now be discussed with reference to the accompanying drawing figures. While the below example is specifically directed to an endoscope used during an ERCP procedure, it is to be understood that this discussion is merely illustrative of one procedure amenable to use of the device of the present invention. This discussion is by no means limiting and one skilled in that art will appreciate that the device disclosed may be suitable for use in any number of surgical procedures and fall within the scope of the present invention.

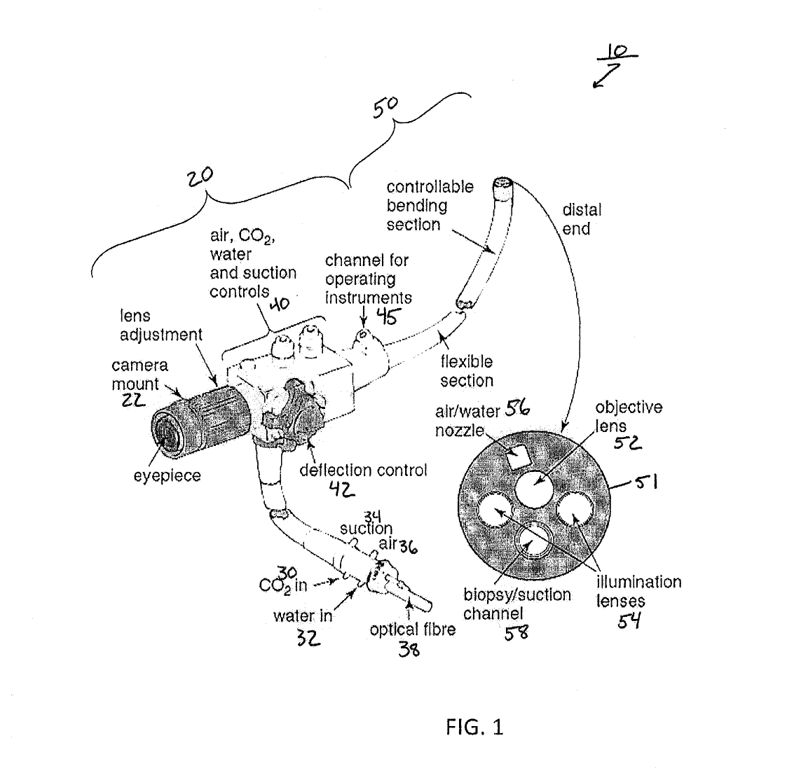

Turning now to FIG. 1, an endoscope 10 typically used in the art has a handle portion 20 and a probe portion 50. Handle portion 20 has an eyepiece/camera mount 22 used for viewing images captured during an examination. Inputs for gas 30, water 32, suction 34, air 36 and optical fiber 38 are directed into the handle and are controlled via control unit 40. Deflection control knob 42 allows the doctor to manipulate the distal end of the probe in three dimensions thereby positioning the probe orifice at the desired location. Handle 20 is further equipped with a channel port 45 for insertion of operating instruments used during an endoscopic procedure. Probe portion 50 is generally comprised of a bundle of tubes and/or operative features housed within a seal-tight flexible member 51 and has an external diameter roughly the size of a large multivitamin. As shown in the expanded view of the distal end of probe portion 50, probe portion 50 terminates with an objective lens 52 which is in communication with optical fiber 38 for the transmission of images. The lens allows the doctor to view the inner surfaces of the body as the endoscope is being passed through the body. At least one illumination lens 54 is provided to supply light to the objective lens 52. An air/water nozzle 56 is also provided and is in fluid communication with the gas input 30 and water input 32. Air/water nozzle 56 permits the doctor to inject a small stream of air or water to clear an area around the objective lens so that the doctor is afforded a clearer view of the surface. Additionally, channel port 45 terminates at a channel opening 58 through which endoscopic tools exit the probe inside the body.

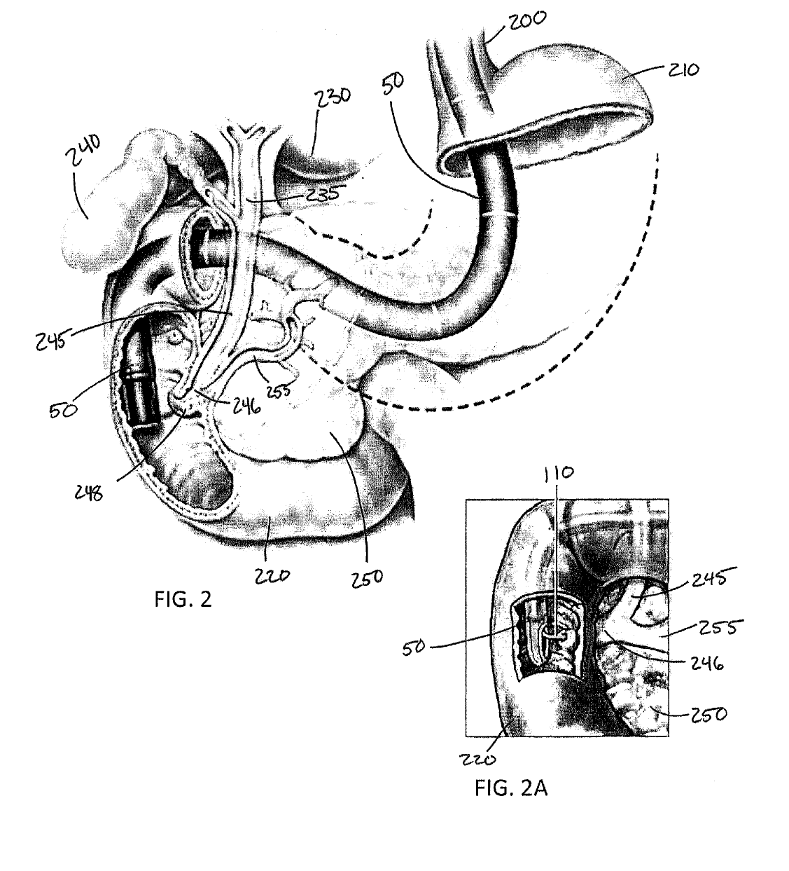

With regard to FIGS. 2 and 2A, a schematic of an ERCP procedure is shown. Endoscope probe portion 50 is passed from a patient's mouth through esophagus 200 into and beyond the stomach 210 to rest within the duodenum 220 near the papilla of Vater, which is the opening of the small intestine leading to the pancreatic 255 and bile 245 ducts. Pancreatic duct 255 injects enzymes produced by pancreas 250 into the small intestine. Hepatic duct 235 extends from the liver 230 and combines with the duct from gallbladder 240 to form common bile duct 245. A small plastic catheter (cannula) 110 is passed through an open channel of the endoscope through the sphincter of Oddi 248 which opens into ampulla 246, and into the bile ducts and/or the pancreatic duct. Contrast material (dye) is then injected and X-rays are taken of the bile ducts and the pancreatic duct. Alternatively, a guide wire accompanies the catheter with the guide wire directed into the ampulla 246. The guide wire has a finer gauge than the catheter and is more flexible and more easily directed by the doctor. Once the guide wire is in place, the doctor “locks” the guide wire in position by securing the external portion of the wire proximate instrument channel 45. Ideally, only one guide wire is needed as each tool is loaded onto the wire and directed to the proper site and then removed from the guide wire once that tool's function has been completed. Thus, a single guide wire directs multiple tools rather than each tool being manipulated within the ampulla or duct of interest. The guide wire allows for quicker examinations while decreasing patient irritation and the potential for pancreatitis—the irritation of the pancreas which can be a side-effect of undergoing ERCP.

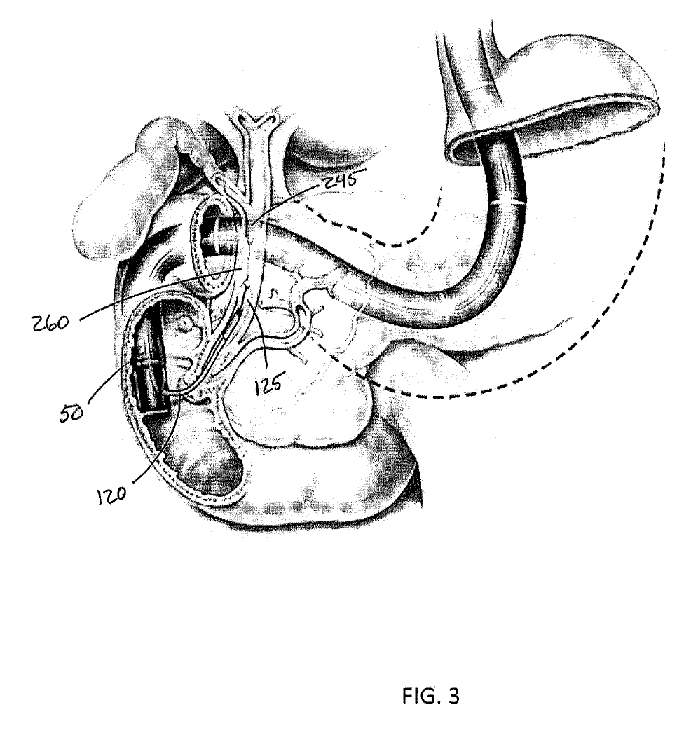

As shown in FIG. 3, an exemplary diagnosis from an X-ray study shows a blockage or constriction 260 of common bile duct 245. There are a number of conditions, malignant or benign, that can cause strictures of the bile duct. Pancreatic cancer is the most common malignant cause, followed by cancers of the gallbladder, bile duct, liver, and large intestine. Noncancerous causes of bile duct stricture include: injury to the bile ducts during surgery for gallbladder removal; pancreatitis; primary sclerosing cholangitis (an inflammation of the bile ducts that may cause pain, jaundice, itching, or other symptoms); gallstones; radiation therapy; and even blunt trauma to the abdomen. To determine the malignancy of the stricture, a doctor may perform a biopsy of the tissue blocking the duct. A biopsy probe is inserted into channel 45 and passed through channel opening 58 where it is directed by a guide wire and the walls of the common bile duct to the blockage site. The guide wire may have been inserted during catheterization, as discussed with reference to FIG. 2, or the biopsy probe may have been preloaded with its own dedicated guide wire which is inserted and directed as discussed above. The doctor then manipulates the biopsy probe to “pinch off” a small portion of the blockage tissue. The probe, with tissue sample attached, is then removed from the endoscope for outside study of the tissue sample (i.e. in a tissue culture lab). The guide wire is then removed if no longer required, or is secured in place to aid in placement of yet an additional endoscope tool.

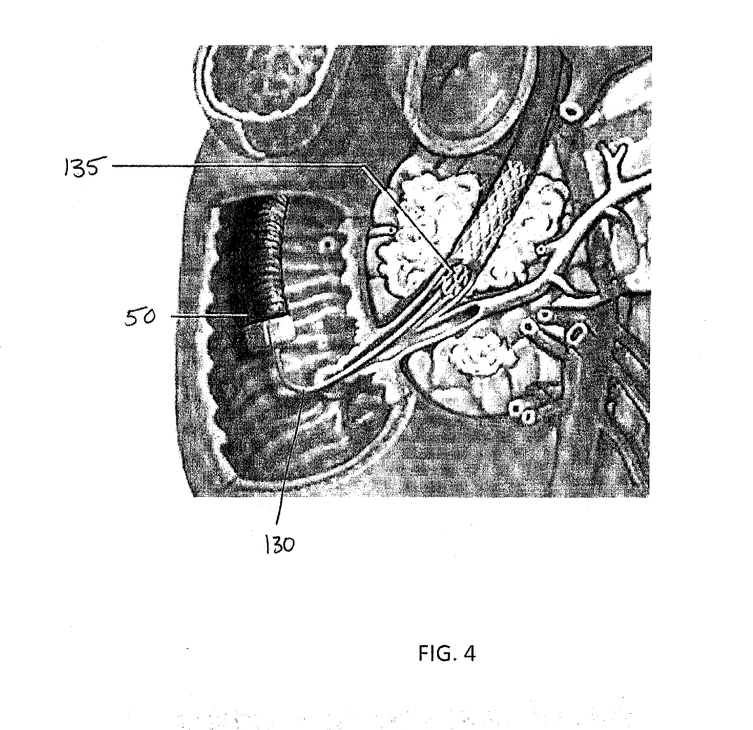

If a constriction of the common bile duct is found, a doctor may choose to implant a stent to relieve the blockage and allow proper draining of bile from the common bile duct into the small intestine. As shown in FIG. 4, a stent probe 130 is inserted within the endoscope probe 50 and directed, via a guide wire (preloaded or already in place), to the blockage site. Stent 135 is then secured in place to prop open the walls of the duct. Optionally, a balloon catheter, not shown, may also be employed to dilate the duct prior to placement of the stent 135.

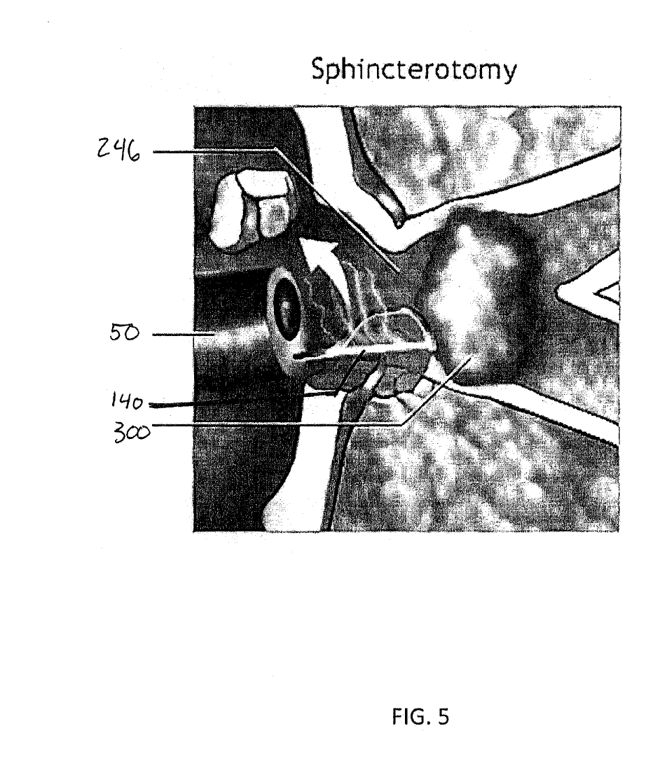

As shown in FIG. 5, an alternative diagnosis during an ERCP procedure uncovers the presence of a gallstone 300. If the gallstone is blocking the papilla of Vater, one technique to alleviate the obstruction is to conduct an endoscopic sphincterotomy. A sphincterotomy probe 140 is passed down the endoscope 50 where it is directed to the sphincter of Oddi by use of a guide wire. Sphincterotomy probe 140 has a heated electric wire which ablates and enlarges the papilla of Vater such that the gallstone 300 can then pass through the enlarged opening and into the small intestine thereby removing the blockage. An endoscopic sphincterotomy may also be employed prior to any of the procedures described with regard to FIGS. 2 through 4 depending on the size of the opening of the papilla and the need to pass instruments through the opening and into the ampulla or ducts.

Important to the present invention, each of the above described procedures may be conducted in one patient visit. For instance, a patient undergoing an ERCP test may have a blocked or narrow papilla of Vater which needs to be enlarged through an endoscopic sphincterotomy. Once the papilla is opened enough to permit passage of a catheter, video and/or X-ray images may further show a constriction of the ampulla or a duct, whether it is the common bile duct, hepatic duct, or pancreatic duct. The doctor may then perform a biopsy to retrieve a tissue sample of the obstruction to determine the malignancy of the cells which are causing the blockage. Once the biopsy is complete, the doctor may then insert a stent within the duct to alleviate the blockage and allow normal flow of the fluids from the blocked organ to the small intestine. In each of these procedures, a different probe is inserted into the endoscope and directed to the operation site. Movement of the each of the probes is accomplished through manipulation of the active member (i.e. catheter orifice for proper injection of contract; direction of the heated wire during sphincterotomy; placement of the stent within the blockage) by way of a guide wire. Thus, during the course of the test, multiple wires will be employed at different times. To facilitate increased ease for the doctor while minimizing risks to the patient, it is imperative that each of these wires be secured and maintained, to the greatest degree possible, in a sterile environment.

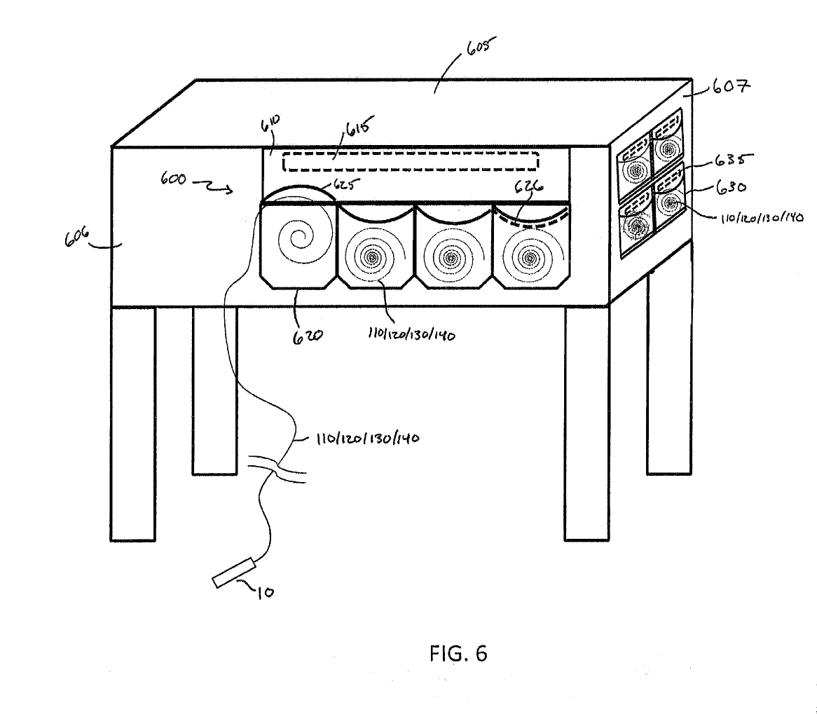

Turning now to FIG. 6, an embodiment of a disposable holder for specialized medical device materials, such as the endoscopic wires and stents discussed above in reference to FIGS. 1-5, is referenced generally by numeral 600. Disposable holder 600 is generally comprised of a fabric back panel 610 to which is affixed a plurality of pouches 620. One or both of back panel 610 and pouches 620 may be constructed from any suitable material, preferably a medical grade material such as used for making sterilizer bags and pouches that are permeable to gases but impermeable to micro-organisms. Pouch 620 may further be equipped with an optional resealable closure 626. Examples of closure 626 include a pressure sensitive adhesive, hook-and-loop, a groove-and-flange and the like.

Prior to a surgical procedure involving the use of surgical wires, catheters, tubes or other similar materials, disposable holder 600 may be releasably secured by fastener 615 to a surgical cart or table having a top 605, a front surface 606 and side surfaces 607. In a preferred embodiment, fastener 615 is a moderate tack adhesive able to stably adhere the holder to the front surface 606 without dislodging when subjected to the pulls and tugs associated with removing and replacing the materials stored within the pouch 620, yet also of low enough tack to allow for quick and easy removal of the holder once the procedure is completed. Prior to use, the adhesive may be covered by a contact paper that is easily peeled off at time of use. While described as an adhesive, fastener 615 may also be any other suitable fastener such as, but not limited to, hook and loop, snaps, buttons, clips, clasps, zippers, or a mechanical fastener such as a staple, screw or tack. It is understood that should any alternative fastener be used, front surface 606 would need to be fitted with the appropriate corresponding fastener so as to form a complete coupling. Within each pouch 620 is housed a surgical tool or accessory thereof, such as the endoscopic tools 110/120/130/140 as described above. Ideally, each pouch is a sterile environment thereby housing sterile tools.

In an alternative embodiment 630, back panel 610 is absent and each of stand-alone pouches 630 is equipped with a fastener 635 along its back face. In this alternative embodiment, only pouches housing necessary tools are mounted to the surgical cart, such as along a side surface 607. Similar to pouches 620, pouch 630 may be equipped with an optional resealable closure. The use of single pouches allows doctors and nurses to equip the operating room with only those tools that will be needed during the procedure thereby minimizing contamination of unused tools and requiring less sterilization time and chemicals to clean the tools between procedures. Fewer pouches (and tools) also reduces clutter within the surgical room during a surgical procedure making for a safer operating room.

When conducting a procedure (for example, ERCP), should a doctor require a specific tool (i.e. a sphincterotomy probe), the assistant removes the tool from its pouch and passes the tool to the doctor (in this example, for insertion within the endoscope), being careful that any wires do not contact the floor or other non-sterile surface. Continuing the example, once the sphincterotomy is completed, the probe is removed and wound upon itself for replacement within its respective pouch 620. If the guide wire used to direct the probe remains within the endoscope, the assistant ensures that the external end of the guide wire does not touch the floor. Should another tool be required, it is simply removed from its respective pouch and positioned over the guide wire for insertion within the probe. Alternatively, if this second tool is preloaded with its own guide wire, the tool and guide wire are passed within the probe to the site of interest. The first guide wire (i.e. that one used with the sphincterotomy probe) may be retained within the probe or can be removed. If the first guide wire is removed, it too is coiled and stored with the sphincterotomy probe within sphincterotomy probe pouch. In this manner, each probe is segregated and maintained in a designated pouch. This creates a clutter-free surgical environment while minimizing the potential for entanglement of wires and endoscopic tools, while also promoting the maintenance of sterility among the tools. A similar scenario would enfold for alternative medical procedures, such as an angiography procedure requiring a catheter, guide wires and delivery means for contrast agent/dye, and for angioplasty requiring a catheter, guide wires, stent, and other necessary devices.

Although the present invention has been described in considerable detail with reference to certain aspects thereof, other versions are possible. Therefore, the spirit and scope of the appended claims should not be limited to the description of the aspects contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

1. A disposable holder for storing and organizing medical procedure materials wherein the disposable holder is mounted to a surface during a medical procedure, the disposable holder comprising:
a back panel having a front face and a back face;
a plurality of pouches mounted onto the front face of the back panel, each pouch including an opening to access a respective medical procedure material; and
a fastener secured to the back face of the back panel wherein the fastener is adapted to mount the disposable holder to the surface.

2. The disposable holder according to claim 1 wherein the fastener is a moderate tack adhesive.

3. The disposable holder according to claim 1 wherein each of the plurality of pouches is constructed of medical grade material that is permeable to gases but impermeable to micro-organisms.

4. The disposable holder according to claim 1 wherein the opening includes a resealable closure.

5. The disposable holder according to claim 4 wherein said resealable closure is selected from the list comprising a pressure sensitive adhesive, a hook-and-loop fastener, and a groove-and-flange fastener.

6. A disposable holder for storing and organizing a medical procedure material wherein the disposable holder is mounted to a surface during a medical procedure, the disposable holder comprising:
a pouch having a back panel and a front panel wherein side edges and bottom edges of the panels are sealed together thereby defining a pouch interior and wherein the top edges are openable to access said pouch interior to retrieve said medical procedure material; and
a fastener secured to the exterior surface of the back panel wherein the fastener is adapted to mount the disposable holder to the surface.

7. The disposable holder according to claim 6 wherein the fastener is a moderate tack adhesive.

8. The disposable holder according to claim 6 wherein each of the plurality of pouches is constructed of medical grade material that is permeable to gases but impermeable to micro-organisms.

9. The disposable holder according to claim 6 wherein said top edges include a resealable closure.

10. The disposable holder according to claim 9 wherein said resealable closure is selected from the list comprising a pressure sensitive adhesive, a hook-and-loop fastener, and a groove-and-flange fastener.
